(19) United States
(12) Patent Application Publication
(10) Pub. No.: US 2026/0325199 A1
(43) Pub. Date: Oct. 22, 2026

(54) METHOD, APPARATUS, AND SYSTEM FOR REMOVAL OF PAPULES

(71) Applicant: Lombardi Institute of Dermatology, PLLC, Tampa, FL (US)

(72) Inventor: Milan Matthew Lombardi, Tampa, FL (US)

(73) Assignee: Lombardi Institute of Dermatology, PLLC, Tampa, FL (US)

(21) Appl. No.: 19/104,331

(22) PCT Filed: Aug. 18, 2023
(86) PCT No.: PCT/US2023/030557

§ 371 Date: Feb. 17, 2025

Related U.S. Application Data
(60) Provisional application No. 63/371,871, filed on Aug. 18, 2022.

(51) Int. Cl. A61B 18/14; A61B 18/00

(52) U.S. Cl. CPC A61B 18/14; A61B 2018/00005; A61B 2018/00315; A61B 2018/0047; A61B 2018/00761

(57) ABSTRACT

A system and method of removing a papule by using an electrosurgical device or any process using radio frequency alternating current to heat tissue. The device can utilize an on-and-off staccato like pattern and can have a sharp pencil tip that is monopolar and monoterminal, that utilizes a low current, high voltage, high frequency current with dampened oscillations. The method can include applying a numbing agent to a body part for treatment, pre-cooling the body part for a period of time, contacting the papule with a distal tip of a terminal probe of an electrosurgical device or any process using radio frequency alternating current to heat tissue, applying current to the papule in a short burst to desiccate the papule, and stopping the current short of complete destruction of the papule to allow healing. The method can be repeated at set intervals.

### CROSS-REFERENCE TO RELATED APPLICATIONS

This application is a U.S. National Stage Application under 35 U.S.C. § 371 of PCT Application No. PCT/US2023/30557, filed Aug. 18, 2023, which claims the benefit of U.S. Provisional Patent Application No. 63/371,871, filed Aug. 18, 2022, each of which are incorporated herein by reference as if repeated in their entirety.

### SUMMARY

In one aspect, the present disclosure involves a procedure for removal of papules, including, for example, pearly penile papules, such as from men. In one aspect, the procedure safely, quickly, effectively, consistently, and without pain removes papules and provides long-term cosmetic and functional outcome.

In another aspect, the present disclosure involves a procedure that includes steps to ensure universal patient comfort during the procedure, quick healing after the procedure, and utilization of affordable technology, which results in consistent exceptional results.

### BRIEF DESCRIPTION OF DRAWINGS

The features and advantages of the example embodiments described herein will become apparent to those skilled in the art to which this disclosure relates upon reading the following description, with reference to the accompanying drawings, in which:

FIGS. 1A and 1B show a process of an exemplary process for removing papules.

### DETAILED DESCRIPTION

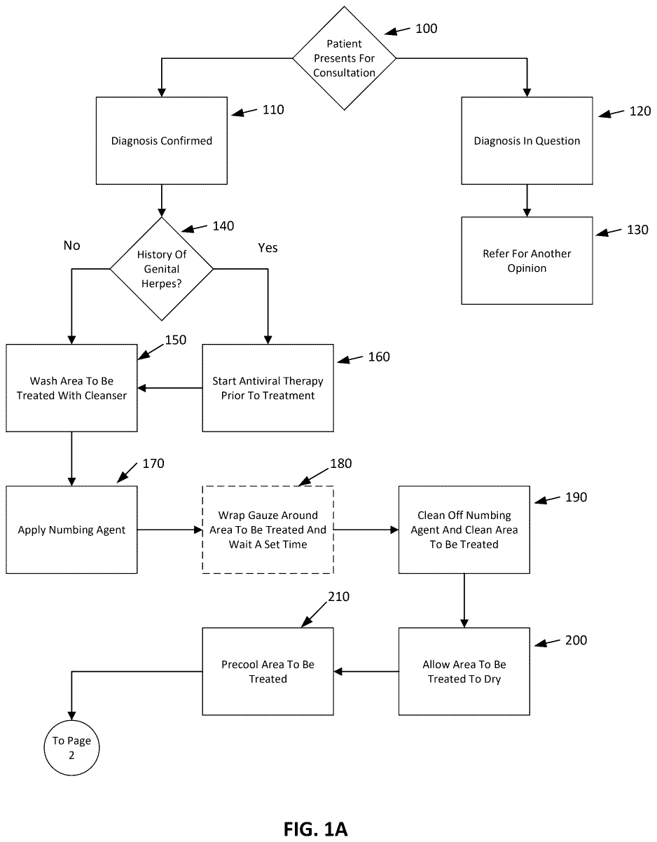

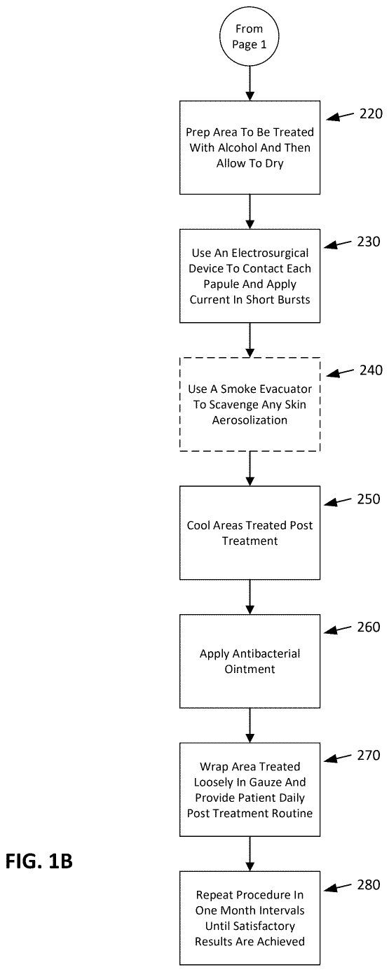

In one exemplary embodiment, the procedure begins with anesthesia, such as with a patient self-anesthetizing the area to be treated, such as by using lidocaine ointment under occlusion, including via saran wrap or a condom for a period of time, e.g., 30 minutes. The anesthesia can, alternatively be applied or additionally be supplemented, by a nurse or other practitioner. The anesthesia is then removed, generally by wiping off or otherwise cleaning the area to be treated. Thereafter, an electrosurgical device or any process using radio frequency alternating current to heat tissue is used in an on-and-off staccato like pattern. In one exemplary embodiment, an electrosurgical device or any process using radio frequency alternating current to heat tissue is utilized with a sharp pencil tip that is monopolar and monoterminal, that utilizes a low current, high voltage, high frequency current with dampened oscillations. Although other patterns can be utilized, a staccato like pattern, such as is referenced in this exemplary embodiment, desiccates the visible, as well as deeper, less visible, lesion(s) of concern, while taking care not to treat or cause collateral damage to the surrounding uninvolved tissue. An exemplary process is shown in the attached flowchart at FIGS. 1A and 1B.

As shown in FIGS. 1A and 1B, the exemplary process begins at 100 where a patient presents for consultation and/or confirmation of diagnosis. As step 100 is a decision diamond, the process proceeds either to step 110 or step 120. At step 110, the diagnosis is confirmed. At step 120, the diagnosis is in question. If the diagnosis is in question after step 120, the process continues to step 130 where the patient is referred to a board certified dermatologist or urologist. If the diagnosis is confirmed at step 110, the process proceeds to a decision diamond 140 to decide whether the patient has a history of herpes. If the patient does not have a history of herpes, the process proceeds to step 150. If the patient has a history of herpes, the process proceeds to step 160 where antiviral therapy is started one to three days prior to treatment or as soon as possible if treatment is imminent. At step 150, if foreskin is present, the foreskin is retracted and the entire penis is washed with sodium hypochlorite based cleanser or other appropriate antimicrobial cleanser. The process proceeds to step 170 where, with the foreskin still retracted, if present, a topical numbing agent is applied. At step 180, the foreskin is replaced, if present, and loose gauze is wrapped around the penis. The process allows the numbing agent to work, such as by waiting for a period of time, e.g., thirty minutes, before proceeding. At step 190, the foreskin is retracted, if present, and the ointment is cleaned off, such as with dry gauze. The process proceeds to clean glans penis, area to be treated, and distal penis shaft with chlorhexidine wash or other appropriate antimicrobial wash and, again, with alcohol. The process then proceeds to step 200 where the alcohol is allowed to dry for a period of time, e.g., one minute. The process proceeds to step 210 where the area to be treated is precooled. The precooling can be performed, for example, with forced cool air, with a chiller or other chilling device, or with a pre-chilled compress with a surface that has been sterilized, or a similarly effective method, for a period of time, e.g., one minute. If utilized, the compress can be formed from, for example, stainless steel, sapphire, or another material effective for cooling.

The process continues from FIG. 1A to FIG. 1B and from step 210 to step 220 where alcohol prep of the area to be treated is repeated and the area is allowed to dry for a period of time, e.g., one minute. The process then proceeds to step 230 where, with the foreskin retracted, if present, a magnifier or magnification system is used generally for closer inspection of the surface. Here, the penile shaft is held steady, e.g., with the non-dominant hand, such as with the thumb, fourth and fifth fingers, and, using the remaining digits, such as first and second fingers to manipulate and stretch areas of glans penis to be treated. Then, an electrosurgical device or any process using radio frequency alternating current to heat tissue that is monoterminal and monopolar, which is set to a low amperage setting, for example, in at least one embodiment, a setting of 0.5 mA-4.0 mA is used, with a setting of 1.5-2.5 mA used in another embodiment. In at least one exemplary embodiment, a terminal probe that is sufficiently tapered and pointed at the distal tip to make contact with each individual papule and not the surrounding skin is used. Here, current is applied to an individual papule, one at a time, in short bursts, for example, in at least one embodiment, a time scale from 0.1 s-3.0 s is used, with a time scale of 0.25 -2.0 s used in another embodiment, based on the mA current settings, in order to achieve sufficient desiccation of lesion which will appear as whitening and melting of the lesion. Additionally, the process involves stopping short of complete destruction of the lesion that would leave open, eroded skin present. The process then proceeds to step 240 where, working circumferentially, the steps above are completed for all lesions. In at least one exemplary embodiment, a smoke evacuator is used to scavenge any skin aerosolization. The process then proceeds to step 250 where, after all lesions have been treated, the treated areas are cooled post treatment in a manner similar to the manner the area was pre-cooled prior to treatment, as discussed hereinabove. The process then proceeds to step 260 where, an antibacterial ointment is applied, such as mupirocin ointment, to treated areas generously and the foreskin, if present, is replaced. The process then proceeds to step 270 where, the penis is wrapped loosely in gauze and the patient is provided post treatment instructions. For example, the post treatment instructions could include washing the area daily with soap and water, applying antibacterial ointment, and avoiding any friction to the area for 10 days. The process then proceeds to step 280 where, the entire procedure is repeated in one month intervals until final satisfactory results are achieved.

In the drawings, like numerals indicate like elements throughout. Certain terminology is used herein for convenience only and is not to be taken as a limitation on the present disclosure. The terminology includes the words specifically mentioned, derivatives thereof and words of similar import. The embodiments illustrated below are not intended to be exhaustive or to limit the disclosure to the precise form disclosed. These embodiments are chosen and described to best explain the principles of the disclosure and its application and practical use and to enable others skilled in the art to best utilize the disclosure.

The present disclosure can be understood more readily by reference to the instant detailed description, examples, and claims. It is to be understood that this disclosure is not limited to the specific systems, devices, and/or methods disclosed unless otherwise specified, as such can, of course, vary. It is also to be understood that the terminology used herein is for the purpose of describing particular aspects only and is not intended to be limiting.

The instant description of the disclosure is provided as an enabling teaching of the disclosure in its best, currently known aspect. Those skilled in the relevant art will recognize that many changes can be made to the aspects described, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the instant description is provided as illustrative of the principles of the present disclosure and not in limitation thereof.

As used herein, the singular forms “a,” “an” and “the” include plural referents unless the context clearly dictates otherwise. Thus, for example, reference to a “body” includes aspects having two or more bodies unless the context clearly indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

Although several aspects of the disclosure have been disclosed in the foregoing specification, it is understood by those skilled in the art that many modifications and other aspects of the disclosure will come to mind to which the disclosure pertains, having the benefit of the teaching presented in the foregoing description and associated drawings. It is thus understood that the disclosure is not limited to the specific aspects disclosed hereinabove, and that many modifications and other aspects are intended to be included within the scope of the appended claims. Moreover, although specific terms are employed herein, as well as in the claims that follow, they are used only in a generic and descriptive sense, and not for the purposes of limiting the described disclosure.

What is claimed is:

1. A method for removing a papule, the method comprising:
applying a numbing agent to a body part for treatment;
pre-cooling the body part for a period of time;
contacting the papule with a distal tip of a terminal probe of an electrosurgical device or any process using radio frequency alternating current to heat tissue;
applying current to the papule in a short burst to desiccate the papule;
wherein the current is stopped short of complete destruction of the papule.

2. The method of claim 1 further comprising:
post-cooling the body part.

3. The method of claim 2 further comprising:
applying ointment after the post-cooling of the body part.

4. The method of claim 1 wherein, prior to applying the numbing agent, the method comprises:
confirming a diagnosis that the papule should be removed.

5. The method of claim 4 wherein, prior to applying the numbing agent, the method comprises:
confirming whether a patient has a history of herpes.

6. The method of claim 5, wherein, if the patient has a history of herpes, an antiviral therapy is started prior to treatment.

7. The method of claim 1, wherein, prior to applying the numbing agent, the body part is cleaned.

8. The method of claim 1, wherein prior to pre-cooling the body part for the period of time, the method comprises:
cleaning off the numbing agent.

9. The method of claim 8, wherein, after cleaning off the numbing agent, the body part is treated with alcohol and then allowed to dry.

10. The method of claim 1, wherein the pre-cooling is performed by forced cool air, a chiller, or pre-chilled compress.

11. The method of claim 10, wherein the period of time is about one minute.

12. The method of claim 10, wherein the pre-chilled compress is formed from stainless steel or sapphire.

13. The method of claim 1, wherein a magnifier or magnification system is used during the applying of the current.

14. The method of claim 1, wherein the distal tip is tapered and pointed to make contact with the papule and not a surrounding area.

15. The method of claim 1, wherein the current is applied in short bursts.

16. The method of claim 15, wherein the short bursts are about 0.25 to 2 seconds.

17. The method of claim 1, wherein a smoke evacuator is used when the current is applied in order to scavenge any skin aerosolization.

18. The method of claim 1, wherein the method is repeated on the body part in one month intervals.

19. The method of claim 1, wherein the body part is a penis.

20. The method of claim 19, wherein foreskin is retracted prior to applying the current.
